(19) United States
(12) Patent Application Publication
(10) Pub. No.: US 2010/0178313 A1
(43) Pub. Date: Jul. 15, 2010

(54) Implantable Degradable Biopolymer Fiber Devices

(75) Inventors: Christian Klein Larsen, Eiksmarka (NO); Therese Andersen, Sande i Vestfold (NO)

Correspondence Address: PATENT ADMINISTRATOR;FMC CORPORATION, 1735 MARKET STREET, PHILADELPHIA, PA, 19103, US

(73) Assignee: FMC CORPORATION, Philadelphia, PA (US)

(21) Appl. No.: 12/663,641

(22) PCT Filed: Jun. 13, 2008
(86) PCT No.: PCT/US08/66819

§ 371 Date: Mar. 11, 2010

Related U.S. Application Data
(60) Provisional application No. 60/943,787, filed on Jun. 13, 2007.
(60) Provisional application No. 60/943,800, filed on Jun. 13, 2007.
(60) Provisional application No. 61/013,216, filed on Dec. 12, 2007.
(60) Provisional application No. 61/013,223, filed on Dec. 12, 2007.

(51) Int. Cl. A61K 9/00; A61K 38/00; A61K 38/22; A61K 38/08; A61K 38/10; A61P 43/00

(52) U.S. Cl. 424/426; 514/2; 514/18; 514/17; 514/16; 514/15

(57) ABSTRACT

Degradable fibers that include biopolymers, as well as implantable devices including one or more fibers made from degradable biopolymers, e.g., alginate, chitosan, hyaluronans or their derivatives. The devices provide a combination of degradability and biocompatibility with physical properties suitable for use of the devices as implants. Exemplary devices are fastening devices including one or more biopolymer fibers. The use of such degradable biopolymers minimizes or eliminates the need for a second surgery to remove the implant, thereby eliminating the additional cost and potential complications of such a second surgery and should reduce the likelihood of secondary fractures resulting from the stress-shielding effect or the presence of screws holes that serve as stress concentrators. Methods for the fabrication of the degradable biopolymer fibers of the present invention are also provided, as well as methods for the fabrication of implantable degradable devices of the present invention which contain one or more degradable biopolymer fibers.

### FIELD OF THE INVENTION

The present invention is directed to implantable degradable biopolymer fiber devices, as well as to methods of manufacture and use thereof.

### BACKGROUND OF THE INVENTION

Use of implantable degradable fixative devices, such as devices made of erodible/enzymatically degradable biopolymers, e.g., alginate, chitosan, hyaluronate or their derivatives will minimize or eliminate the need for a second surgery to remove the implanted device. It may also eliminate or reduce the occurrence of complications during a potential second surgery and it should reduce the likelihood of secondary fractures resulting from the stress-shielding effect or the presence of screw holes that serve as stress concentrators. Use of degradable fixative devices will also eliminate the cost related to secondary surgeries since such devices need not be removed once implanted.

Some bioabsorbable products on the market consist of polymers that release degradation products not favorable for the healing area. Examples of bioabsorbable materials used in existing degradable fixation products are polyhydroxyacids, e.g. polylactides, polyglycolides and their copolymers, and polycarbonates. The degradation products from polyhydroxyacids induce an unfavorable lowered pH value around the healing area. An effect of this is prolonged inflammatory response and reversal of an initial healthy tissue response.

Alginate is a widely used material for tissue regeneration and cell immobilization, for example, in the form of hydrogels or porous scaffolds. Chitosan is also a common biopolymer in implantable biomaterials, and it is known from the literature to enhance osteogenesis and is of special interest for scientists working in the orthopedic area. Hyaluronate is a biopolymer naturally occurring in the human body as the second most abundant after collagen in the extracellular matrix (ECM). Hyaluronate is also an important component of articular cartilage and it contributes to tissue hydrodynamics, movement and proliferation of cells, and participates in a number of cell surface receptor interactions.

Zhong et al., U.S. Pat. No. 6,368,356, discloses medical devices comprising hydrogel polymers with ionic crosslinks having improved mechanical strength with at least two segments that degrade in vivo at different rates. The different segments differ in their type of crosslinking, ionic versus covalent, or, alternatively the segments are not biodegradable.

Luzio et al., U.S. Pat. No. 5,531,716, discloses medical devices subjected to triggered disintegration. The medical devices comprise ionically crosslinked polymers that have sufficient mechanical strength to serve as a stent, catheter, cannula, plug or constrictor. The methods presented to create the materials involve forcing the crosslinkable polymer through a shaping die into a crosslinking bath, use of molding compositions with the crosslinkable polymer in solution, or use of materials wherein the crosslinking ion is in an insoluble or slowly soluble form, and additives are included to cause dissolution of the crosslinking ion. The created gel can be further developed, crosslinked and/or shaped by soaking in a solution of a crosslinking ion. Also required is a triggered disintegration of the device induced by administering or triggering release of an agent which displaces the crosslinking ion through the diet, parenteral feeding or an enema, administering the agent directly onto the device in an aqueous solution or encapsulating the agent in the device.

Teoh et al., U.S. patent application publication no. US 2007/0083268 A1, discloses bioabsorbable plug implants and methods for bone tissue regeneration. The bioabsorbable plug implants comprise a first portion and a second portion extending outwardly from the first portion, the first and second portions being formed from expandable material. It is mentioned that any bioabsorbable material known in the art suitable for the construction of the plug implant can be used. In the method for bone tissue regeneration of the device may be inserted into a defect or gap of a bone.

Ashammakhi and Törmälä in International patent application publication no. WO 2005/009496, disclose an implant device for bone fixation or augmentation in a mammalian body to enhance the mechanical strength of a fracture. Some of these devices may contain fibers of bioabsorbable polymers.

U.S. Patent application publication no. US 2006/0193769 (Nelson) discloses drug releasing biodegradable fibers for delivery of therapeutics. These fibers may be formed into matrices or scaffolds and may comprise biopolymer hydrogels.

U.S. Pat. No. 6,372,248 (Qin et al.) discloses a dehydrated hydrogel incorporating a plasticizer and fibers which contain cations for cross-linking the dehydrated hydrogel. These hydrogels may be formed into films for wound dressing and are designed to absorb large quantities of water.

U.S. Patent application publication no. US 2007/0077271 discloses medical devices coated with a fast dissolving biocompatible coating which may be a non-crosslinked water soluble salt of alginic acid, hyaluronic acid or chitosan, wherein the coating is readily dissolvable in at least one mammalian body fluid.

Alginate is a widely used material for tissue regeneration and cell immobilization. Alginate is used, for example, in the form of hydrogels or porous scaffolds. Chitosan is also a common biopolymer in implantable biomaterials, and it is known from the literature to enhance osteogenesis and is of special interest for scientists working in the orthopedic area. Hyaluronate is a biopolymer naturally occurring in the human body as the second most abundant after collagen in the extracellular matrix (ECM). Hyaluronate is also an important component of articular cartilage, and it contributes to tissue hydrodynamics, movement and proliferation of cells, and participates in a number of cell surface receptor interactions.

Accordingly, there remains a need for improved implantable devices which can be degraded to harmless materials within the body while permitting such devices to accomplish useful functions including, for example, load-bearing functions.

### SUMMARY OF THE INVENTION

In a first embodiment, the present invention relates to degradable fibers made from at least one biopolymer and modified biopolymers such as modified polysaccharides and to implantable devices including at least one fiber made from a degradable biopolymer or modified biopolymers, e.g., alginate, chitosan, hyaluronans or modified versions thereof. The devices provide a combination of degradability and biocompatibility with physical properties suitable for use of the devices as implants. Exemplary devices are fixative devices including one or more biopolymer fibers. The use of such degradable biopolymers minimizes or eliminates the need for a second surgery to remove the implant, thereby eliminating the additional cost and potential complications of such a second surgery and should reduce the likelihood of secondary fractures resulting from the stress-shielding effect or the presence of screws holes that serve as stress concentrators.

In other embodiments, the present invention relates to methods for the fabrication of the degradable biopolymer fibers of the present invention, as well as to methods for the fabrication of devices including such fibers.

In other embodiments, the present invention relates to method for the fabrication of implantable degradable devices of the present invention which contain one or more degradable biopolymer fibers.

### BRIEF DESCRIPTION OF THE DRAWINGS

FIG. 1 (i.e., FIGS. 1A and 1B) shows pictures of one embodiment of the present invention (i.e., a screw) that was prepared in Example 1 hereinbelow.

### DETAILED DESCRIPTION OF THE INVENTION

The present invention is directed to an implantable degradable device comprising biopolymer fiber, as well as to methods of manufacture and use thereof. Biopolymers include polymers that are produced by living organisms, as well as materials derived from a polymer produced by a living organism by some type of synthetic modification of the material that was produced by a living organism. Some examples of such synthetic modification processes are described below. Classes of suitable biopolymers include polysaccharides, polypeptides and polypeptides covalently bonded to polysaccharides in any desired ratio.

As used herein, “degradable” refers to the device of the present invention wherein the device naturally disappears over time in vivo from or in accordance with any biological or physiological mechanism, such as, for example, erosion including bioerosion, degradation, dissolution, chemical depolymerization including at least acid- and base-catalyzed hydrolysis and free radical induced depolymerization, enzymatic depolymerization, absorption and/or resorption within the body. As a result, the degradable devices of the present invention do not require surgical removal in either adults or children. Eliminating the insertion of a non-biologic implant will have several advantages. Removal of the implant and a second surgery will not be necessary, and the establishment of a new growing tissue will not be inhibited. Additionally a degradable implant will save both time and costs.

The use of biopolymers as the degradable material for fibers and devices including the fibers will be beneficial compared to the commonly used synthetic polymers due to surface properties. As the surfaces of many synthetic polymers are hydrophobic this will typically hinder cell growth, whereas the hydrophilic biopolymers may promote cell proliferation and cell differentiation. Additionally, further modification of synthetic polymers may be necessary to provide the required functional groups.

Examples of the biopolymer fiber that may be used in the present invention are fibers that comprise alginates, chitosans, hyaluronans, modified versions thereof, and mixtures thereof. None of these biopolymers are known to cause unfavorable conditions for formation of new tissue. Degradable medical attachment devices of the invention comprising biopolymer fibers from any of the above listed biopolymers such as alginate fibers and chitosan fibers are suitable for attachment or regrowth of damaged tissue. The fiber content of the devices of the present invention may range, for example, from about 5 to about 100% and, more preferably, fiber-containing devices will contain from about 30 to about 100% fiber. The fibers typically contain at least 85% solids.

Ultrapure biopolymers having sufficient purity to render such biopolymers suitable for implantation without causing inflammatory responses should be used. Ultrapure biopolymers have a reduced content of endotoxins. By reduced endotoxin content, it is meant that the endotoxin, protein and heavy metal content of the biopolymers used to prepare the device and the endotoxin content of the medical device together must not exceed, for example, the U.S. Food and Drug Administration recommended endotoxin contents for implantable medical devices. The current regulatory guidelines establish that a device may not release to the patient more than 350 EU (5 EU/kg).

Alginates are salts of alginic acid. Alginates are a family of non-branched binary copolymers of 1→4 glycosidically linked β-D-mannuronate (M) and α-L-guluronate (G) monomers. The relative amount of the two uronate monomers and their sequential arrangement along the polymer chain vary widely, depending on the origin of the alginate. Alginate is the structural polymer in marine brown algae such as Laminaria hyperborea, Macrocystis pyrifera, Lessonia nigrescens and Ascophyllum nodosum. Alginate is also produced by certain bacteria such as Pseudomonas aeruginos and Azotobacter vinelandii. The ratio of mannuronate and guluronate varies with factors such as seaweed species, plant age, and part of the seaweed (e.g., stem, leaf). The uronic acid residues are distributed along the polymer chain in a pattern of blocks, where homopolymeric blocks of G residues (G-blocks), homopolymeric blocks of M residues (M-blocks) and blocks with alternating sequence of M and G units (MG-blocks) co-exist. The alginate molecule cannot be described by the monomer composition alone. Composition and sequential structure together with molecular weight and molecular conformation are the key characteristics of alginate in determining its properties and functionality.

Examples of the alginate include alginate having a G content greater than 50%, a G content greater than 60%, a G content greater than 70%, a G content greater than 80%, and a G content greater than 90% and mixtures thereof. Additional examples include an alginate having an M content of greater than 50%, an M content greater than 60%, an M content greater than 70%, and an M content greater than 80% and mixtures thereof. Mixtures of alginates having such G content and M content may also be used. For example, it has been found that decreasing the G content of the alginate relative to the M content produces stronger dried devices. Examples of the alginate include alginates having a molecular weight less than 500 kDa. Suitable alginates have a molecular weight greater than 4,000 Daltons.

When alginate is used as the biopolymer, the gelling cations that may be present will be exchanged with non-gelling ions over time, which gradually makes the polymer soluble in natural solvents present in the body, e.g. water. Soluble alginate will be depolymerized by acid- or base-catalyzed hydrolysis, or free radicals. When the alginate has been depolymerized to a lower molecular weight, it is naturally excreted from the body through the kidneys. When chitosan is used as the biopolymer, it will undergo enzymatic hydrolysis mediated by lysozymes present in mammalian saliva, tears, blood serum and in interstitial fluids. Additionally, anions will be exchanged over time if the chitosan is ionically cross-linked. When hyaluronate is used as the biopolymer it will be enzymatically degraded from hyaluronidases present in mammalian tissues and cells, blood plasma, synovial fluid and urine. The device of the invention can be designed to retain the needed strength for a sufficient time period after insertion and then gradually disappear, e.g., degrade/bioabsorb, as the healing process progresses. None of the degradation products of the biopolymers used in the present invention are known to induce any undesired effects in newly formed tissue or within the human or mammalian body.

As used herein, “100% saturation” of the alginate molecule is considered to be 1 mole divalent cation per 2 moles uronic acid (D-mannuronic acid and L-guluronic acid). Alginates create heat stable gels at physiologic conditions when divalent cations as e.g. calcium, strontium or barium are present.

The crosslinking agents for the biopolymers of the invention may contain divalent or trivalent cations or water soluble salts containing phosphate or citrate, and are preferably present in an amount sufficient to saturate the biopolymer to 0.001% to 200%. Suitable cations may include, but are not limited to, calcium, barium, lead, manganese, cobalt, nickel, iron, zinc, copper, aluminum, citrate, holmium and phosphate.

Chitin is a linear polysaccharide comprising β-(1→4)-linked 2-acetamido-2-deoxy-D-glucopyranose (GlcNAc) and 2-amino-2-deoxy-D-glucopyranose (GlcN). Chitin is present in nature as the structural element in the exoskeleton of crustaceans (crabs, shrimp, etc.). Chitosan is a fully or partially N-deacetylated derivative of chitin. Chitin consists nearly entirely of β-(1→4)-linked 2-acetamido-2-dexoy-D-glucopyranose (GlcNAc). Commercially, chitosan is made by alkaline N-deacetylation of chitin. The heterogeneous deacetylation process combined with removal of insoluble compound results in a chitosan product which possesses a random distribution of GlcNAc and GlcN-units along the polymer chain. The amino group in chitosan has an apparent pKₐ-value of about 6.5 and at a pH below this value, the free amino group will be protonized so the chitosan salt dissolved in solution will carry a positive charge. Accordingly, chitosan is able to react with negatively charged components, it being a direct function of the positive charge density of chitosan. The positive charge gives the chitosan bioadhesive properties.

Chitosan deacetylation protects the polymer from enzymatic degradation. Thus, varying the degree of chitosan deacetylation can modify the rate of biodegradation of implanted chitosan-containing devices by lysozymes. Chitosans with higher degrees of deacetylation are also more resistant to random depolymerization by acid hydrolysis due to a protective effect of the positive charge. Examples of the chitosan include chitosan with a degree of deacetylation in the range of 40% to 100%. Suitable molecular weights are in the range 10 kDa to 1000 kDa. Blends of alginates and chitosans may be particularly advantageous since the anionic alginates may interact with the cationic chitosans to form a more stable matrix of material. Also, chitosans can be coated with alginates to modify the degradation properties of the material, in which case chitosan deactylation could be replaced, by, for example, the provision of an alginate coating on a chitosan fiber.

Hyaluronate is a linear polymer that is composed of glucuronate and N-acetylglucosamine monomers linked alternately by β(1→3) and β(1→4) glycosidic bonds. The polymer is an important part of the extracellular matrix. For example, it is a major component of the synovial fluid. It was found to increase the viscosity of fluids and along with lubricin, it is one of the fluid's main lubricating components as the coiled structure can trap approximately 1000 times its weight in water. Hyaluronate is also an important component of articular cartilage and a major component of skin, where it is involved in tissue repair.

Commercially available hyaluronate is usually made by fermentation from e.g. Streptococcus zooepidemicus or derived from avian (chicken or rooster) combs. The available molecular weights of commercially available hyaluronates are less than 5000 kDa and will be suitable for this invention.

The biopolymers can be tailored by selection of moieties and concentrations added to form modified biopolymers. To modify or alter properties or functionalities of the biopolymers, such as, for example cross-linking capability, solubility, rate of biodegradability and the ability to bind, specific cells, pharmaceuticals or peptides may also be included.

Modified polysaccharides may include synthetic analogues of polysaccharides formed by covalent bonding onto the polysaccharide, polysaccharides modified by enzymatic modification, e.g. epimerization of alginates, as well as oxidation of polysaccharides. Covalent bonding may be used to attach a variety of materials including peptide sequences, sugar units, hydrophobic groups such as thiol groups and alkyl chains.

Modified polysaccharides may be covalently linked to a polymer backbone. Preferred linked polysaccharide groups are alginates or modified alginates containing functional sites. The polysaccharide, particularly alginate, when present as side chains on the polymer backbone, may include side chains at the terminal end of the backbone, thus being a continuation of the main chain. The modified polysaccharides and modified alginates exhibit controllable properties depending upon the ultimate use thereof. One example of modified alginates can be found in U.S. Pat. No. 6,642,363 (Mooney et al.), the disclosure of which is hereby incorporated by reference for a description of such materials and methods for making them. Mooney et al. discloses modified alginates, methods of preparation and uses thereof such as cell transplantation matrices, preformed hydrogels for cell transplantation, non-degradable matrices for immunoisolated cell transplantation, vehicles for drug delivery, wound dressings and replacements for industrially applied alginates.

Modified polysaccharides such as modified alginates may also be prepared by covalently bonding to add a biologically active molecule for cell adhesion or other cellular interaction. Crosslinked modified alginates with the biologically active molecules in a three-dimensional environment are particularly advantageous for cell adhesion, thus making such alginates useful as cell transplantation matrices. In some embodiments, the modified alginate is a biologically active molecule for cell adhesion or other cellular interaction, which is particularly advantageous for maintenance, viability, proliferation, mobility and differentiation.

Modified alginates can also be prepared using an approach combining chemical and enzymatic techniques. One example of this approach can be found in International patent application publication no. WO 06/051421 A1. The starting alginate can have varying amounts of M and G which may be grouped in varying structural arrangements of MM, GG, and/or MG blocks. A chemical reaction step will lead to substituents reacted on the M and G residues of the alginate as applicable. The enzymatic step will change the amount of M and G in the alginate by converting a desired number of M residues to G residues. For example, the amount of G is increased by converting MM blocks to MG or GG or converting MG blocks to GG.

Coupling of the cell adhesion molecules to the biopolymer can be conducted utilizing synthetic methods which are in general known to one of ordinary skill in the art. A particularly useful method is by formation of an amide bond between the carboxylic acid groups on the alginate chain and amine groups on the cell adhesion molecule. Other useful bonding chemistries include those discussed in Hermanson, Bioconjugate Techniques, p. 152-185 (1996), particularly by use of carbodiimide couplers, DCC and DIC (Woodward's Reagent K). Since many of the cell adhesion molecules are peptides, they contain a terminal amine group for such bonding. The amide bond formation is preferably catalyzed by 1-ethyl-3-(3-dimethylaminopropyl)carbodiimide (EDC), which is a water soluble enzyme commonly used in peptide synthesis.

In some embodiments, the biopolymer, e.g. alginate comprises one or more cell adhesion peptides covalently linked thereto. In some embodiments, the alginate comprises one or more cell adhesion peptides covalently linked thereto. Suitable cell adhesion peptides comprising RGD include, but are not limited, to Novatach RGD (NovaMatrix, FMC BioPolymer, Oslo, Norway) and those disclosed in U.S. Pat. No. 6,642,363, which is hereby incorporated by reference in its entirety. Peptide synthesis services are available from numerous companies, including Commonwealth Biotechnologies, Inc. of Richmond, Va., USA. Chemical techniques for coupling peptides to the alginate backbones may be found in U.S. Pat. No. 6,642,363.

Examples of modified alginates may be found in, “Dual Growth Factor Delivery and Controlled Scaffold Degradation Enhancement in vivo Bone Formation by Transplanted Bone Marrow Stromal Cells,” Simmons, C. A. et al., Bone 35 (2004), pp. 562-569, and “Regulating Bone Formation via Controlled Scaffold Degradation,” E. Alsberg, et al., J. Dent. Res. 82 (11), pp. 903-908 (2003).

Examples of oxidized alginates can be found, for example, in European patent application publication no. EP 0 849 281.

Blends of hyaluronate and chitosans may be particularly advantageous since the anionic hyaluronate may interact with the cationic chitosans to form a more stable matrix of material. In one aspect of the present invention, anionic and cationic biopolymers are mixed or blended to form the biopolymer used in the devices of the present invention. It has been found, for example, that blending of anionic and cationic biopolymers at varying ratios can be employed to customize at least the strength, degradation and swelling properties of the resultant device. Depending on the particular use desired for a particular fixative, it may be beneficial to customize these properties for that use. Blends typically contain from about 25 to about 75% by weight of the cationic polymer, based on the total weight of the cationic and anionic polymers, and, more preferably, contain from about 35 to about 65% by weight of the cationic polymer, most preferably, from about 45 to about 55% by weight of the cationic polymer, based on the total weight of the cationic and anionic polymers.

Fibers may contain any suitable amount of biopolymer, for example, at least 85% by weight of biopolymer, at least 90% by weight of biopolymer, at least 95% by weight of biopolymer, or 100% by weight of biopolymer. Higher biopolymer contents typically result in stronger fibers which are more stable against degradation. The fibers may have a diameter, for example, in the range of 100 nm to 1 mm. During the manufacture of the fibers, tissue inductive materials can optionally be included in the biopolymer solution.

The fibers used to manufacture the device can be of similar type in relation to diameter, biopolymer used, type of crosslinking and degree of crosslinking, or mixtures of different types of fibers, which vary in one or more of these properties, may also be used. It is preferred to employ fibers that are ionically crosslinked due to the presence of crosslinking cations in body fluids. Combinations of fibers from cationic and anionic biopolymer can be used to modify the stability of the device as ionic interactions will take place between the polymers and further stabilize the device. The fibers may be used as wet fibers to fabricate the device, prior to drying the wet fiber. In such case, wet fibers typically comprise from 0.1-15% by weight of biopolymer such as alginate, based on the total weight of the fiber. The formed device may subsequently be dried after fabrication.

The present invention is also directed to a method of making the implantable degradable fastening device of present invention comprising the step of forming the device from the at least one biopolymer fiber.

The biopolymer fibers of the present invention can be prepared using any known technique. Also, a variety of different types of fibers may be prepared including, for example, non-crosslinked fibers, ionically crosslinked fibers or covalently crosslinked fibers. The degree of crosslinking can be stoichiometric or sub-stoichiometric, as desired to obtain the particular properties sought for a particular device or part of a device. In this manner, partial crosslinking can be employed as one method for providing a controlled rate of degradation of the fiber or the device. The rate of degradation or resorption of the biopolymer system may be controlled by varying the degree of cross-linking and the molecular weight of the components using any suitable technique, one illustrative technique being described in, for example, Kong, et al “Controlling rigidity and degradation of alginate hydrogels via molecular weight distribution,” Biomacromolecules, 2004, 5, 1720-1727, the disclosure of which is hereby incorporated herein by reference for a description of this technique.

A plasticizer may also be employed in the device of the present invention. When a plasticizer is employed in the device of the present invention, an amount of 0.01% to 70% by weight of the biopolymer may be employed. More preferably, 0.01% by weight to 50% by weight of the plasticizer, based on the weight of the biopolymer may be employed. Alternatively, an amount of 0.01% by weight to 25% by weight of plasticizer, based on the weight of the biopolymer, may be employed. Suitable plasticizers include, for example, at least one of glycerin, sorbitol, ethylene glycol, propylene glycol, and polyethylene glycol. Larger amounts of plasticizer may also be employed to increase the degradation rate of the fibers and/or devices made therefrom.

In some embodiments, the devices and/or fibers of the present invention may contain degradable biopolymer, as well as one or more of an uncrosslinked degradation controlling agent, an imaging agent, a gelling ion, an alcohol a tissue regenerative additive, a cell adhesion peptide sequence, or a pharmaceutically active agent selected from, but not limited to, a growth factor agent, an antiseptic, an anticoagulant, an antibiotic, an anti-inflammatory, a pain-killer, a chemotherapeutic agent, and an anti-infective agent, a protein or a drug to modify the properties of the fiber and/or device. The device may also contain one or more other therapeutic agents selected from enzymes, transcription factors, signaling molecules, internal messengers, second messengers, kinases, proteases, cytokines, chemokines, structural proteins, interleukins, hormones, pro-coagulants, agents that promote angiogenesis, agents that inhibit angiogenesis, immunomodulators, chemotactic agents, agents that promote apoptosis, agents that inhibit apoptosis, and mitogenic agents.

The cell adhesion peptide sequence may be a biologically active molecule for promoting or causing cell adhesion or other cellular interaction. Combinations of two or more different cell adhesion peptide-linked biopolymers for example in biostructures, beads or hydrogels may provide particularly useful advantages for repairing, reconstructing and treating conditions of tissue. Biologically active molecules for cell adhesion or other cellular interaction are well known and widely recognized and available. U.S. Pat. Nos. 4,988,621, 4,792,525, 5,965,997, 4,879,237, 4,789,734 and 6,642,363, which are incorporated herein by reference, disclose numerous examples. Suitable peptides include, but are not limited to, peptides having about 10 amino acids or less. In some embodiments, cell adhesion peptides comprise RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). Cell adhesion peptides comprising RGD may be in some embodiments, 3, 4, 5, 6, 7, 8, 9 or 10 amino acids in length. When using “RGD peptides”, those peptides containing the RGD motif, such as GGGGRGDY, GGGGRGDSP, GRGDSP, the interaction is dependent upon the way the RGD sequence is presented to the cells, for example, the concentration and/or orientation.

These additional materials may be provided to the device of the present invention in any suitable manner, for example, by being directly mixed into the biopolymer, as part of or as a coating on the device, as a filler in hollow portions of the device as described herein or as a filler contained in a suitable vehicle, e.g. a biopolymer hydrogel, located in hollow portions of the device and/or the fibers used in the device.

Suitably, the device of the invention is sterilized, preferably by γ-irradiation, E-beam, ethylene oxide, autoclaving, alcohol treatment, supercritical CO₂, or contacting with NOₓ gases or by hydrogen gas plasma sterilization. A suitable sterilization should be employed which does not adversely affect the properties of the device in a significant, detrimental manner.

The device may be treated in an aqueous bath comprising at least one of a sequestering agent or non-gelling ion to partly solubilize the biopolymer fibers during manufacture of the device or after the device is shaped and/or fuse the fibers. Suitable sequestering agents for ionically crosslinked fibers made from alginate may include, but are not limited to, EDTA, EGTA, phosphates, citrates, polyphosphates and mixtures thereof. The sequestering agent may be present in an amount of 0.001-10 weight percent, based on the weight of the aqueous bath. Suitable non-gelling ions may be, for example, at least one of sodium, potassium, magnesium, lithium, ammonium or silver.

The device may also be treated, with a sequestering agent or non-gelling ion, in an aqueous bath comprising at least one of a degradable biopolymer, an uncrosslinked degradation controlling agent, an imaging agent, a gelling agent such as a gelling ion, an alcohol a tissue regenerative additive, a cell adhesion sequence or a pharmaceutically active agent selected from, but not limited to, a growth factor, an antiseptic, an anticoagulant, an antibiotic, a protein, an anti-inflammatory, a pain-killer, a chemotherapeutic agent, an anti-infective agent, or a drug to further modify the properties of the device. In some embodiments, the device is treated in a solution of at least one gelling agent to gel the biopolymer and form a continuous, gelled layer. At least one gelling agent may be present in an amount of 0.01-10 weight percent of the aqueous bath. This treatment may be used in combination with one or more of the other treatments discussed above. The treatment(s) may last for up to 24 hours.

The implantable devices of the present invention may be used, for example, in the treatment of diseases and disorders of tissues including, but not limited to, bones, and adjacent tissue such as muscle, cartilage, connective tissue, nerve and vascular tissue. The devices of the invention may also be useful in the repair, reconstruction of bone tissue and treatment of conditions and diseases of bone and adjacent tissues including but not limited to soft tissue, nerve, cartilage in the knee, shoulder, rotator cuff, ligaments and tendons.

The present invention relates to implantable devices comprising biopolymer fiber. The term, “devices” as used herein refers to fixative or fixation devices, as defined elsewhere herein, as well as to other implantable devices used for tissue repair and/or regeneration. The present devices are pre-shaped objects in that these devices are formed into the desired shape prior to implantation. Though some limited amounts of swelling may occur upon implantation, this should not significantly alter the general shape of the pre-shaped devices of the present invention, instead influencing the size of the device.

The implantable device of the present invention may have an elongated body. In some embodiments, the device of the invention may be, for example, a screw, plug, bolt, anchor or pin that can be used for fastening any portion of body tissue (e.g., muscle, bone, cartilage, tendon, etc.) to another. The device of the invention will, because of the fibers, withstand torque forces. A thread design may easily be made on the device as well. When the device of the invention is a screw, it may be a fully-threaded screw, i.e. a screw with threads along the entire length of the device, or it may be a partially threaded screw with threads located only on a proximal or distal part of the screw.

The device of the invention can be solid or hollow in one or more parts of the material, or the entire device may be hollow. The device may, for example, comprise a biopolymer based degradable body or core which is surrounded by biopolymer fibers.

The present invention also relates to a method for making a degradable fastening device by forming the device from at least one biopolymer fiber. The device may be formed by a plurality of biopolymer fibers and may include any one or more of the additives or modifications discussed herein. Such devices may include screws, bolts, anchors, plugs, pins, or rods.

In one embodiment of the invention, the crosslinked biopolymer fibers are aligned to form a three-dimensional shaped device. Then the device is treated first in a bath containing a sequestrant for the gelling ions in the fiber (e.g. aqueous EDTA in the case of ionically crosslinked alginate fibers) to remove a portion of the crosslinking ions from the fiber surface, and then in a coagulation bath which may include a plasticizer and crosslinking ions (e.g. divalent cations for alginate) to gel the fiber surfaces together, followed by drying. This bath may be an aqueous bath that includes some alcohol therein. The alcohol may be present in the bath in a sufficient amount to prevent the biopolymer from complete solubilization. In this manner, the device can be modified to include one or more biopolymer or alginate gel layers. This bath may also optionally include one or more biopolymers, non-crosslinked degradation control agents, imaging agents, pharmaceutically active agents, cell adhesion peptide sequences and growth factor agents, as desired. The growth factor agent used in the various methods of the present invention may be selected from bone morphogenic proteins, transforming growth factors (beta), fibroblast growth factors, platelet derived growth factors, vascular endothelial growth factors, insulin-like growth factors, epidermal growth factors and mixtures thereof.

Another embodiment of the invention includes treating the shaped device in an aqueous biopolymer solution. For example, if gelled alginate fibers are present in the device, a treatment in alginate solution will initiate dissolution of the alginate fibers as the gelling ions from the fibers will be shared with the surrounding alginate solution. An exemplary biopolymer solution may be a solution of sodium alginate. This will give a partly gelled alginate hydrogel surrounding the device, which, when dried, will form a film or a coating. Before drying, the device may be treated in an aqueous bath containing gelling ions to further add gelling ions to the coating layer in order to modify the degradation rate and/or swelling properties. The coating layer may also contain any of the other agents discussed above for inclusion in the biopolymer, device or fiber. The biopolymer solutions may optionally contain a plasticizer to reduce brittleness and modify hydration rates.

Treatment with the biopolymer solution may occur at one or more stages of the fabrication process. For example, the device may be treated once with a biopolymer solution to provide a protective coating layer on the exterior of the device. Alternatively, the treatment with biopolymer solution may be carried out after application of each individual fiber layer.

The film may, upon hydration after insertion, swell to fill potential voids between e.g. the bone and the inserted device, to interlock the device. The pressure caused by the swelling may also stimulate the healing of the injured tissue. The film can contain tissue regenerative agents as e.g. growth factors, antibiotics, peptide sequences or drugs. In general, film thickness can be controlled by the concentration of the biopolymer solution, viscosity of the biopolymer solution or the residence time the device is located in the biopolymer solution. When coating layers are added during manufacturing, layers containing different materials can be used to modify, for example, drug release and degradation properties. Such coatings may include, for example, sustained release agents, immediate release agents and delayed release agents. The coating layer is preferably applied on the exterior of the device.

Another embodiment of the device of the invention involves winding biopolymer fibers around a core made from one or more biopolymers. The core can be a shaped core and may be degradable, as defined herein. The core can be made, for example, from an extruded biopolymer paste that is either air-dried or freeze dried, a molded dried biopolymer paste or a milled dried biopolymer paste. Alternatively, the core can be a mesh. The biopolymers used are preferably alginates, chitosans, hyaluronates, their modified derivatives or mixtures thereof.

This core may be porous and have a degradation rate different from the surrounding fibers in order to facilitate tissue ingrowth. The core may include a material favorable for tissue regeneration, for tissue growth or both. Winding the fibers around the core may provide additional strength, retard degradation and reduce swelling of the core due to hydration.

The core may be fabricated by the application of pressure to a partially hydrated biopolymer or modified biopolymer containing material to form a degradable pre-shaped core. Pressure may be applied, for example, by molding, extrusion or other suitable processes. The application of pressure may compress, compact or densify the material. Also, some de-aeration of the material may occur as a result of the application of pressure due to compression of the material. It has been observed that in some embodiments using biopolymers, the application of pressure may cause a transition to a more transparent material, perhaps due to more uniform hydration of the material as a result of compression. Thus, when applying pressure to biopolymers, in some embodiments, sufficient pressure should be applied to provide a substantially homogeneous material which is transparent. By substantially homogeneous is meant that the hydration of the material is nearly uniform throughout the material once sufficient pressure has been applied.

The material may be partially or fully hydrated prior to application of pressure with higher degrees of hydration being preferred for some embodiments since a higher degree of hydration appears to provide a material of greater strength in the formed device. The core preferably has a dry solids content of 85-100%, more preferably, 88-95% by weight, based on the total weight of the core.

The core may optionally be dried. Any conventional drying process may be used although, in some instances, controlled drying may be desirable for a variety of reasons such as controlling the shape and/or size of the final core. Preferred drying methods include air drying and freeze drying. It has been found that use of a particular drying process may influence the final properties of the core and thus selection of a drying process may be employed for core customization. For example, the strength and porosity of the device can be altered by selection of a particular drying process.

The water content of the material prior to application of pressure to the core material may vary over a wide range. In practice, the water content may depend on such factors as the degree of hydration that is desired for a particular material, as well as the flowability of the material that may be required for processing. Thus, water contents of 40-65% by weight are preferred for the materials of the present invention that are fed to the step of applying pressure since at these water contents, the material is best-suited for processing and can be handled in an efficient manner. Use of lower water contents may be a way to reduce shrinking of the product, upon drying.

Other embodiments of the device may involve weaving or layering at least one fiber onto a core made from one or more biopolymers. Combinations of winding, weaving and layering may also be employed. The winding, weaving or layering may be done in one direction, or it may be done in more than one direction, as desired. For example, the winding, weaving and/or layering may provide at least one biopolymer fiber at an angle of 0.01-180 degrees, relative to the axial cross-section of the device. Also, a plurality of fibers may be associated to form a bundle of fibers, and the bundle of fibers may be formed into a device by winding, weaving, layering or any combination thereof, around a core made from one or more biopolymers. A bundle of fibers may contain, for example, 2 to 10,000 individual fibers.

The core may be removed from the device after winding, weaving and/or layering. Alternatively, the core, made from one or more biopolymers, may be omitted and the device may be formed from one or more fibers, bundles of fibers or combinations thereof, by winding, weaving, layering or any combination thereof to provide a device of the desired shape. In another embodiment, the core may be replaced by or formed from freeze-dried biopolymer such as freeze-dried alginate. Preferably, the core contains alginate in non-crosslinked form, partially crosslinked form or crosslinked form, or as a mixture of two or more alginates having different degrees of crosslinking.

Whether or not a core made from a biopolymer is employed, the fibers and/or bundles of fibers may be twisted together or placed substantially parallel relative to one another, as desired. The spacing between parallel fibers and/or bundles of fibers may be varied to provide the desired properties of the device. In certain embodiments, the maximum spacing between a fiber surface and the adjacent surface of the nearest adjacent fiber should be no more than 1 μm in order to provide advantages in the device such as degradation control, control of swellability and/or improved device strength. Use of a small spacing between fibers may reduce or prevent degradation of a core material of the device, for example, or retard degradation of the device by presenting a smaller effective surface area to materials which may cause degradation. Small fiber spacing also increases strength and reduces the swellability of the device. Thus, the fiber spacing can be employed as a means to control a variety of the properties of the device, either alone, or in combination with the various other methods of controlling the properties of the device described elsewhere herein. Also, the parallel fibers and/or bundles of fibers may include fibers located in a single plane or in multiple planes. Also, fibers and/or bundles of fibers may be made by spinning one or more threads and/or fibers.

Another embodiment of the invention includes a device wherein a hollow or partially hollow fiber based screw; plug, bolt, anchor, rod, or pin is filled with a material such as a biopolymer-based hydrogel. This hydrogel can contain osteoinductive materials, osteoconductive materials, demineralized bone or tissue regenerative additives such as, for example, growth factors, cell adhesion peptide sequences, osteoprogenitor cells, fibroblasts, cartilage, bone cells, including osteoblasts and osteoclasts, blood vessel cells, including vascular endothelial and perivascular endothelial cells. any genetically engineered cells that secrete therapeutic agents, such as proteins or hormones for treating disease or other conditions, genetically engineered cells that secrete diagnostic agents and stem cells. These materials can also be used as a filler in devices of the present invention without incorporation into a hydrogel. The hydrogel can be manufactured by any method known in the art. Preferably the gel is set after or during filling of the hollow device. Setting of the gel may be induced by, for example, a temperature change or use of a self-gelling alginate system as described by Melvik et al. (WO 2006/044342 A2), the disclosure of which is hereby incorporated by reference for the purpose of describing the self-gelling alginate.

When hollow or gel-filled devices are employed, the implant mass will be reduced and the surface area will be larger. This may be used to further increase the substitution rate of bone. This may allow regeneration of tissue from both inside and outside of the device. If the tissue structure is created from the inside of the device structure, the loss of mechanical strength of the device as it degrades may be less important.

The devices of the present invention may, in some embodiments have a rotationally symmetric shape. In some embodiments, the biopolymer fiber is used to build a structure of a woven or non-woven type in the device. The degradation properties of the device may be customized by one or more of the additives, treatments and/or structures described above such that the device may immediately begin to degrade, may exhibit sustained degradation or may have delayed degradation. Also, various parts of the device may be tailored to have different degradation rates and/or immediate, sustained or delayed degradation.

The devices of the present invention may be load-bearing. Thus, some devices of the present invention will have sufficient strength and structural integrity to bear a load in use. By “load-bearing” is meant that the device is fabricated to have sufficient strength and/or structural integrity to bear a load that will be exerted on the device once it is implanted. Load-bearing may refer to a variety of different properties of the device such as its ability to withstand compressive, tensile, torsional and bending forces. A particular device may be able to withstand different levels of these various forces, depending on what is required for the particular use for which that device is destined.

The devices of the present invention may be used as fixatives. The terms, “fixation” and “fixative” refer to devices that are used to position or fix tissue in a desired position, location, orientation or attach or position tissue relative to other tissue, e.g. by attaching two tissues together or supporting two tissues in relationship to one another, including, but not limited to by attachment to the tissue, support of the tissue, or a combination thereof. Fixation of tissue does not necessarily require a load-bearing device and thus in some case, fixatives will not be load-bearing when implanted. For example, in the case of a plug, the plug may be implanted to ensure that materials are maintained in place during a healing period, in which case the plug may not have to bear a load. In another example, the plug may be used to provide a substrate into which a load bearing device may be incorporated, e.g. a plug with a load-bearing screw threaded into it.

The fixative may also be load-bearing and could be a screw which threadably engages tissue such as bone. In another example, the fixative can be a plug which fills a gap or hole in a tissue or fills corresponding gaps or holes in two or more tissues to position the tissues relative to one another. Fixation devices or fixatives include, but are not limited to fastening devices.

Another aspect of the device of the present invention is that it is degradable. Thus, over a period of time, the device should degrade by one or more of the various mechanisms described above. Preferably, the device degrades over a period of 1-6 months, and more preferably, over a period of 2-4 months, or longer. In such case, the device should maintain its important characteristics (e.g. ability to bear a load) during the time period specified. The degradation rate of the device can be tailored using many of the fabrication methods, treatment processes, materials, structures and combinations thereof, which have been described herein.

The swelling properties of the devices of the present invention may be customized for a particular use. The devices may swell when exposed to bodily fluids. In some embodiments, a relatively high swellability may be desired, for example, to provide a friction fit or force fit between the implant and the tissue. A plug implanted in a hole or gap in a bone may be retained in position by swelling of the plug to provide a tight fit with the bone. Such a plug could be used as a substrate for fixation of a screw or other device in the body. In some embodiments, swelling may be beneficial for triggering tissue regeneration by exertion of pressure on the area where tissue regeneration is desired. In other embodiments, a relative low swellability may be desired such that the device substantially retains its original size when implanted. In most embodiments a swellability of not more than 25% of the original size of the device, is desired. More preferably, for devices requiring lower swellability, swellability may be from 0% to 15%, and most preferably from 0% to 10%.

The swellability of the device can be influenced, for example, by coating a core of the device with fibers in order to retard swelling. Swelling can also be influenced by the method of making the device, the biopolymers used to make the device, post treatment processes and drying methods. In this manner, the swelling properties can be customized for a particular device or application, as desired.

The present invention is now described in more detail by reference to the following examples, but it should be understood that the invention is not construed as being limited thereto. Unless otherwise indicated herein, all parts, percents, ratios and the like are by weight.

### EXAMPLES

### Example 1

An implantable fastening screw of the present invention was made as follows. Thin calcium alginate fibers were spun up and down around a mold, e.g. a thin needle, until the desired thickness was obtained. Some of the thin fibers were twisted, and spinning the twisted fibers upwards made threads. The resultant threaded screw was transferred to a solution of 50 mM citrate for 2-5 minutes to make the surface of the fibers somewhat soluble by sequestering calcium ions. Other sequestering agents such as EDTA may also be used. The screw was then kept in a solution of 50 mM calcium chloride with 1% glycerin for 2 to 5 minutes to make the screw stronger by gelling the fibers together. The screw was then dried in room temperature or in a drying oven. The mould was removed when the screw was dry.

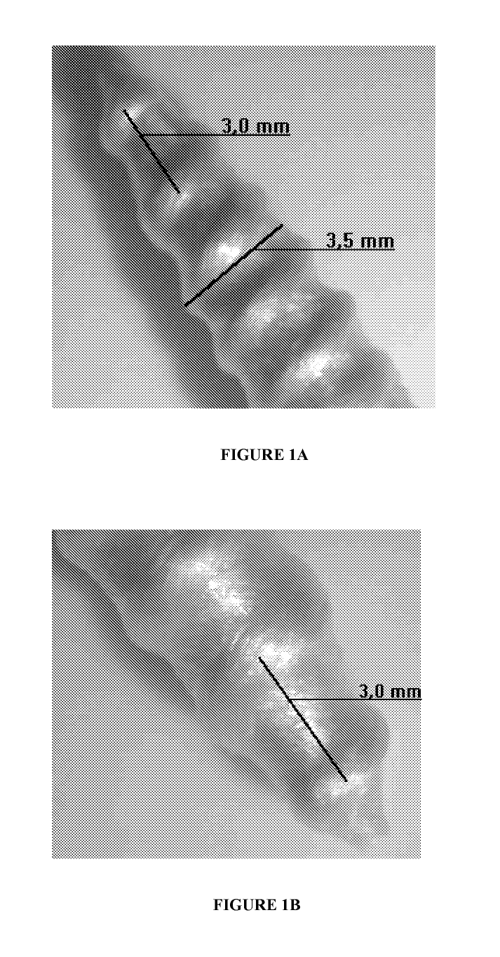

Swelling studies were performed in a model physiological solution, consisting of 142 mM sodium ions and 2.5 mM calcium ions, for 24 hours. The screw's diameter did not increase, but the length increased by about 5-10% as a result of swelling. The screw appeared almost the same as before swelling, without a strength reduction, but somewhat more flexible. Pictures of the screw are shown in FIG. 1. (i.e., FIGS. 1A and 1B).

### Example 2

A plug made of alginate fibers was made by winding a planar bundle of 1000 monofilaments once upwards and once downwards around a 1 mm diameter mold. After fabricating these two layers, the plug was dipped in a 10% (w/w) solution of a low molecular weight sodium alginate for 10 seconds. After withdrawal of the plug, excess alginate solution was removed, and three fiber bundles were attached in the longitudinal direction to increase tensile strength. This was followed by another upwards and downwards winding of fibers. The process of dipping in alginate solution, attachment of longitudinal fibers and another upwards and downwards winding of fibers was repeated twice. Then, the plug was dipped in a 3% (w/w) solution of a high molecular weight alginate for 10 seconds, and left for 2 minutes for excess alginate solution to rinse off. The plug was then submerged in a solution of 4.5% (w/w) CaCl₂*2H₂O and 10% glycerol for 5 minutes. The mold was removed, and the plug dried at room temperature. The plug had a length of 46.7 mm and diameter of 3.8 mm.

A texture analyzer was used to measure tensile strength. The plug was fastened between two plates mounted on the texture analyzer. The plug was then stretched at 0.1 mm/sec, and the force at 0.5 mm stretching distance and max distance was recorded.

| Distance (mm) | Force (kg) |
|---|---|
| 0.5 | 1.90 |
| 2.23 | 30.93 |

The plug had a maximum tensile strength of 30.93 kg, and this peak was reached when the plug had been stretched 2.23 mm The plug was very resistant to compression and stretching in the longitudinal direction.

While the invention has been described in detail and with reference to specific embodiments thereof, it will be apparent to one skilled in the art that various changes and modifications can be made therein without departing from the spirit and scope thereof.

### Example 3

This example describes how to make a bolt from cross-linked calcium alginate fiber with a dry alginate gel coating. The example further shows the strength measurement of a dry bolt and a bolt that is partly hydrated in a model physiological solution.

A bolt was made from alginate fibers by winding a bundle of 5000 high-G alginate monofilaments up and down tightly around a needle (diameter: 1 mm, length: 5 cm). The windings were repeated about three times in each direction until the diameter of the bolt was about 5.6 mm. Then the bolt was placed in a 3% aqueous alginate solution (PRONOVA UP LVG, 1% viscosity: 44 mPas, F_{G}: ˜0.7) for 10 minutes. During this treatment it was seen that a gel layer was created around the bolt. This gel layer was created due to diffusion of calcium ions present in the fibers now available to gel the alginate solution surrounding the bolt. By this treatment the fibers on the surface of the bolt are partly dissolved and the bolt is coated with an alginate gel layer. To strengthen the coating layer the bolt was transferred into a gelling bath comprising 5% CaCl₂*2H₂O and 0.5% glycerol for 5 minutes. The needle was removed and the bolt was placed in the gelling bath. After gelling, the diameter of the bolt was about 7.4 mm. The bolt was dried under ambient conditions uncovered on the laboratory bench for at least two days. The diameter of the dry bolt was then about 6.2±0.9 mm. The dried bolt was about 24.9±3.6 mm long and weighed 0.89±0.05 grams (n=10).

To measure the dry strength of the bolt a Texture Analyzer (Stable Micro Systems (SMS), TA-XT2, load cell: 25 kg) and HDP/3PB Three Point Bend Rig was used with a base gap of 15 mm. The mode selected was: “Measure force in compression” and the pre-test speed and test speed were 0.5 mm/s and 0.2 mm/s, respectively. The distance was 10 mm and the trigger force was set to 5 g. The probe was adjusted to hit on the middle of the bolt between the two base legs upon which the bolt was placed. The force was applied vertically on the axis of the bolt. The measured breaking strength was 2480±360 g and the force applied per second before breakage was 240±80 g/s (n=5).

To see how the material swells upon hydration and how the strength suffers, the bolts were placed in 75 ml of Hanks' balanced salt solution (H8264, Sigma-Aldrich Chemie GmbH, Steinheim, Germany) Five bolts were placed in the same 100 ml weighing boat and kept in Hanks' at room temperature for two hours. The diameter and length of the bolts after two hours with swelling were 7.4±0.5 mm and 26.1±0.7 mm, respectively. The strength of the hydrated materials was tested with a Texture Analyzer (SMS, TA-XT21, load cell: 5 kg) and a HDP/BSG Blade Set with Guillotine. The mode selected was: “Measure force in compression” and pre-test speed and test speed were 0.5 mm/s and 0.25 mm/s, respectively. The distance was 10 mm and the trigger force was set to 1 g. The force was applied vertically on the axis of the bolt. The bolts had swelled 6±6% in the radial direction and 6±3% in the axial direction (n=5). The five bolts tested all survived the maximum load of the instrument of 6.4 kg which was obtained after the guillotine had traveled 4.1 mm±0.3 mm.

### Example 4

This example shows how to prepare a bolt from alginate fiber with a core of an extruded dried bolt made from a 1:1 blend of chitosan and hyaluronate. The example further demonstrates how swelling of the core material upon hydration in a model physiological solution is reduced by covering it with alginate fibers.

The extruded bolts were made by blending in a mortar dry powders of 3.21 g hyaluronate (SODIUM HYALURONATE PHARMA GRADE 80, Kibun Food Kemifa Co. Ltd., Tokyo, Japan, dry matter content (DMC): 93.5%) and 3.29 g chitosan (PROTASAN UP CL 210, NovaMatrix, FMC BioPolymer AS, Sandvika, Norway, DMC: 91.09%, degree of deacetylation: >95%). When the powders were blended 8.50 g MilliQ water was added and a homogeneous and hydrated rubber like paste was made with use of the mortar and hand kneading. The moisture content in the paste was 60%. Then the paste was pressed by hand into a metal tube with inner diameter of 6 mm and length of 40 mm. Rubber bolts (2-3 mm thick) were placed in each end of the metal tube and a metal plunger (diameter 5.8 mm) was placed at one end of the tube and the paste was then compressed for 5 minutes using a vice. The rubber bolts were placed at the ends of the tube to be able to exert more compressive force with the vice without extruding the paste. The bolts made from the paste were either dried uncovered under ambient conditions on the laboratory bench for at least two days or placed in a freezer at −18° C. overnight and then vacuum dried for one day. The freeze dried hyaluronate/chitosan bolts had an average diameter of 5.0±0.2 mm and an average density of 0.96±0.12 mg/cm³ (0.18±0.02 g/cm) (n=10). The air dried hyaluronate/chitosan bolts had an average diameter of 4.6±0.2 mm and an average density of 1.23±0.08 mg/cm³ (0.20±0.01 g/cm) (n=10).

The bolts were covered with the same fibers as in Example 3 and the winding of a bundle of fibers was performed as described in the same example, except the windings were made up and down two times in each direction around the bolt. The diameters of the extruded bolts covered by fiber were 6.4±0.3 mm and 6.9±0.3 mm for bolts with freeze dried and air dried cores, respectively. The weights of the extruded material and fiber were 0.71±0.10 g and 0.73±0.05 g for bolts with freeze dried and air dried cores, respectively. Then the bolts were placed in an alginate solution and subsequently a gelling solution as described in Example 3. The resulting thicknesses of the bolts were then 8.3±0.4 mm and 9.1±0.7 mm before drying for the bolts with freeze dried and air dried cores, respectively. After drying uncovered for two days under ambient conditions on the laboratory bench, the diameters and weights of the materials were 6.7±0.7 mm, 0.78±0.09 grams and 6.2±0.6 mm 0.81±0.09 grams for the bolts with freeze dried and air dried cores, respectively.

The strength of the dry materials with and without fibers was tested as described in Example 3 and the average breaking strength, maximum breaking strength and the force applied per second until breakage occurred, are summarized in Table 1. The bolts without fibers were air dried and were not treated in an alginate solution and gelling bath.

TABLE 1
Strength measurements of dry bolts (n = 4-5, ±SD).

| | Average | Maximum | Gradient, |
|---|---|---|---|
| | breaking | breaking | force/second |
| Bolt | strength, [g] | strength, [g] | [g/s] |
| Freeze dried hyaluronate: | 11 500 ± 5 700 | 19 400 | 1 140 ± 530 |
| chitosan (1:1) covered | | | |
| with alginate fibers | | | |
| Air dried hyaluronate: | 14 700 ± 4 800 | 20 700 | 850 ± 480 |
| chitosan (1:1) covered | | | |
| with alginate fibers | | | |
| Air dried hyaluronate: | 20 000 ± 9 000 | 36 800 | 2 970 ± 640 |
| chitosan (1:1) | | | |

The results presented in Table 1 do not show any significant differences between the materials, but indicate that a solid core material may provide a stiffer and stronger material, when compared to the results obtained in Example 3. The force per second applied during measurement was higher for the material not covered with fibers. This is probably due to small amounts of air between the fibers and because compression of the fibers requires less force than was applied to the extruded bolt.

The materials were partly hydrated and the strength was measured as described in Example 3. All the bolts survived the maximum load of 6.4 kg. Table 2 presents the swelling of the material and the distance the guillotine traveled before maximum load was applied.

TABLE 2
Strength measurements of hydrated materials (n = 4-5, ±SD).

| | Freeze dried | Air dried | |
|---|---|---|---|
| | hyaluronate: | hyaluronate: | |
| | chitosan (1:1) | chitosan (1:1) | Air dried |
| | covered with | covered with | hyaluronate: |
| Property | alginate fibers | alginate fibers | chitosan (1:1) |
| Radial swelling, [%] | −0.5 ± 2.0 | 4 ± 5 | 36 ± 7 |
| Axial swelling, [%] | 6 ± 4 | 12 ± 13 | 7 ± 3 |
| Average breaking | >6 400 | >6 400 | 4 700 ± 1 200 |
| strength, [g] | | | |
| Maximum breaking | >6 400 | >6 400 | 6 000 |
| strength, [g] | | | |
| Distance before | 2.2 ± 0.3 | 2.5 ± 0.3 | 5.1 ± 0.7 |
| maximum load, [mm] | | | |

The fibers reduced swelling in the radial direction, but since the fibers were not wound to cover the ends of the bolts, the bolts swelled more in the axial direction. For the bolts without fibers, the guillotine traveled longer before maximum load was applied. This indicates a more flexible material compared with the fiber coated materials. The use of fibers to cover a core made from an extruded biopolymer will reduce swelling and thereby also reduce hydration rate and degradation rate.

### Example 5

This example shows how to prepare a bolt made from alginate fibers with a hollow predefined shaped core.

A hexagonal key was used as the mold and four lengths of a bundle with fiber were wound up and down around it. The diameter of the hexagonal key was 2.4 mm. The diameter of the bolt was 5.3 mm, the length was 28 mm and it weighed 0.28 g. Then the bolt was placed in an alginate solution and gelling bath as described in Example 3. Then the hexagonal key was removed and the bolt was placed in a 50 mM aqueous solution of sodium citrate with 1% glycerol. This solution will sequester calcium ions and start to dissolve the alginate coating and fibers in the centre of the bolt. After ten minutes of degelling the bolt was transferred to the same gelling solution as used earlier. After five minutes the bolt was removed (weight: 1.09 g, diameter: 6.75 mm and length: 32 mm) and dried uncovered under ambient conditions on the laboratory bench. The centre of the dry bolt had a clear hexagonal shape. The weight, diameter and length were 0.31 g, 5.7 mm and 24.2 mm, respectively.

What is claimed is:

1. An implantable degradable pre-shaped device comprising 5-100% by weight of biopolymer fiber, which device is pre-shaped prior to implantation, wherein a maximum spacing between a surface of a fiber and a closest surface of a nearest adjacent fiber in said device is not more than 1 μm.

2. The device of claim 1, wherein said biopolymer fiber comprises at least 85% by weight of solids and said biopolymer comprises a material selected from the group consisting of alginate, chitosan, hyaluronate, modified polysaccharides and mixtures thereof.

3. The device of claim 1, wherein said plasticizer is present in said fiber in an amount of 0.01% to 70% by weight of the biopolymer.

4. The device of claim 2, wherein said device is a screw, pin, bolt, anchor, rod, or plug.

5. The device of claim 1, wherein said biopolymer fiber further comprises at least one material selected from the group consisting of a plasticizer, at least one degradable biopolymer, gelling agent, uncrosslinked degradation controlling agent, imaging agent, pharmaceutically active agent, tissue regenerative agent, tissue adhesive agent, cell adhesion peptide sequence and growth factor.

6. The device of claim 2, wherein said device comprises at least one coating layer of degradable biopolymer.

7. The device of claim 6, wherein said biopolymer coating layer further comprise at least one material selected from the group consisting of plasticizers, gelling agent, non-gelling ions, uncrosslinked degradation controlling agent, imaging agent, pharmaceutically active agent, tissue regenerative agent, tissue adhesive agent, cell adhesion peptide sequence and growth factor.

8. The device of claim 1, wherein said biopolymer fiber builds up a structure of woven type.

9. The device of claim 1, wherein said biopolymer fiber builds up a structure of non-woven type.

10. The device of claim 1, comprising an elongated body.

11. The device of claim 1 in the form of a screw which may be partially or fully threaded.

12. The device of claim 1, wherein said device comprises at least one coating layer that comprises at least one of sustained release agents, immediate release agents and delayed release agents.

13. The device of claim 1, comprising one or more hollow parts filled with a biopolymer hydrogel.

14. The device of claim 13, wherein said biopolymer hydrogel comprises a material selected from the group consisting of alginate, chitosan, hyaluronate, modified polysaccharides, and mixtures thereof.

15. The device of claim 13, wherein said biopolymer hydrogel further comprises at least one material selected from the group consisting of imaging agents, growth factors, pharmaceutically active agents, tissue regenerative agents, cell adhesion peptide sequences and cells.

16. A method of making an implantable degradable device of claim 1, comprising the step of forming the device from said at least one biopolymer fiber.

17. The method of claim 16 wherein said device is a screw, bolt, anchor, plug, rod, or pin.

18. The method of claim 16, wherein said device is formed by at least one of winding, weaving or layering said at least one biopolymer fiber into a desired shape in at least one direction.

19. The method of claim 16, wherein more than one biopolymer fiber is associated together to form one bundle of biopolymer fibers, wherein said bundle are twisted together or placed in parallel proximity, and said device is formed by at least one of winding, weaving or layering of at least one bundle of biopolymer fiber in at least one direction into a desired shape.

20. The method of claim 16 wherein said device is further treated in an aqueous bath comprising at least one of a sequestering agent, non-gelling ion or biopolymer solution to partly solubilize and fuse the biopolymer fibers.

21. The method of claim 20, wherein said device after removal from the aqueous bath is further treated in a solution of at least one gelling agent to gel the biopolymer to form a continuous gelled layer covering fused biopolymer fibers.

22. The method of claim 21, wherein said aqueous bath or solution also comprises at least one of the members from the group of non-gelling ions, uncrosslinked degradation controlling agents, imaging agents, pharmaceutically active agents, tissue regenerative agents, tissue adhesive agents, cell adhesion peptide sequences and growth factors.

23. The method of claim 16, wherein the method comprises the steps of providing a shaped core and winding, weaving or layering at least one biopolymer fiber on said shaped core to form said device.

24. The method of claim 23, further comprising the step of removing said core from said device to create a partially hollow device.

25. The method of claim 23, wherein said provided core is one of a freeze-dried biopolymer and an air-dried biopolymer.

26. The method of claim 23, wherein said core comprises biopolymer which is not substantially ionically crosslinked.

27. The method of claim 23, wherein said core comprises at least one of the members from the group of degradation controlling agent, imaging agent, pharmaceutically active agent, tissue regenerative agent, cell adhesion peptide sequence and growth factor.
